(19) United States
(12) Patent Application Publication
(10) Pub. No.: US 2026/0199126 A1
(43) Pub. Date: Jul. 16, 2026

(54) THERAPEUTIC DEVICE FOR COLD OR HOT COMPRESS

(71) Applicant: Shenzhen Ruizhang Industrial Co., Ltd., Shenzhen (CN)

(72) Inventor: YUYANG TIAN, Dongguan (CN)

(21) Appl. No.: 19/355,189

(22) Filed: Oct. 10, 2025

(30) Foreign Application Priority Data
Jan. 16, 2025 (CN) 202520112144.X

(51) Int. Cl. A61F 7/00; A61F 7/02

(52) U.S. Cl. CPC A61F 7/0085; A61F 2007/0054; A61F 2007/0277

(57) ABSTRACT

A therapeutic device for cold or hot compress is provided, which includes a box body, a connection pipe, and a refrigeration component. The box body forms a liquid storage compartment, which stores liquid. The refrigeration component is configured to reduce the temperature of the liquid in the liquid storage compartment; the connection pipe is communicated to the liquid storage compartment for transporting liquid in and out of the liquid storage compartment. This application utilizes the refrigeration component to exchange temperature with the liquid in the liquid storage compartment, reducing the temperature of the liquid without the need for manual addition of ice cubes. The liquid in the liquid storage compartment can be communicated to a liquid storage bag through the connection pipe, allowing the liquid to enter the liquid storage bag and then use the liquid storage bag for ice therapy, which is very convenient to use.

### CROSS-REFERENCE TO RELATED APPLICATIONS

This application claims priority to Chinese Patent Application No. 202520112144.X, filed on January 16, 2025, which is hereby incorporated by reference in its entirety.

### TECHNICAL FIELD

The present disclosure relates to the field of ice compress equipment technologies, and in particular, to a therapeutic device for cold or hot compress.

### BACKGROUND

The application background of existing water cold compress products is usually through the operation of adding ice cubes to the water tank, which rapidly cools the normal water to a certain extent. Then, through the water circulation, the cooled water is sent to a designated position. Within a certain period, as the ice cubes dissolve, the water temperature will slowly return to room temperature. For scenarios that require long-term constant temperature use, frequent ice cubes need to be added, and the temperature is also fluctuating. It is quite troublesome to manually add ice cubes for use.

### SUMMARY

The purpose of this application is to overcome the technical deficiencies mentioned above and provide a therapeutic device for cold or hot compress, which can solve problems of inconvenient use of ice cubes in cold compress products in the prior art.

To achieve the above technical objectives, the technical solution of the present application is as follows: a therapeutic device for cold or hot compress, including a box body, a connection pipe, and a refrigeration component, where a liquid storage compartment is formed inside the box body, and liquid is configured to be stored in the liquid storage compartment; the refrigeration component is configured to reduce temperature of the liquid in the liquid storage compartment; the connection pipe is configured to communicate with the liquid storage compartment and transport liquid in and out of the liquid storage compartment.

In some embodiments of the present disclosure, the connection pipe includes a first pipeline and a second pipeline, where one end of the first pipeline is communicated to the liquid storage compartment, and one end of the second pipeline is communicated to the liquid storage compartment.

In some embodiments of the present disclosure, the first pipeline is configured to transport the liquid into the liquid storage compartment, and the second pipeline is configured to transport the liquid from the liquid storage compartment.

In some embodiments of the present disclosure, the therapeutic device for cold or hot compress further includes two first quick connectors, where the other end of the first pipeline is connected to one of the two the first quick connectors, and the other end of the second pipeline is connected to the other one of the two first quick connectors.

In some embodiments of the present disclosure, the therapeutic device for cold or hot compress further includes a liquid pump that is connected to the second pipeline.

In some embodiments of the present disclosure, the therapeutic device for cold or hot compress further includes a liquid storage bag, and the liquid storage bag is connected to two second quick connectors; the two second quick connectors connected to the liquid storage bag are respectively connected to the two first quick connectors of the first pipeline and the second pipeline, so that both the first pipeline and the second pipeline are connected to the liquid storage bag.

In some embodiments of the present disclosure, the therapeutic device for cold or hot compress further includes a heating element configured to heat the liquid in the liquid storage compartment.

In some embodiments of the present disclosure, an opening is provided on a top side of the box body configured to add liquid to the liquid storage compartment.

In some embodiments of the present disclosure, the refrigeration component includes an evaporator, a compressor, and a condenser, and the evaporator is provided in the liquid storage compartment.

Compared with existing technologies, the beneficial effects of this application include using the refrigeration component to exchange temperature with the liquid in the liquid storage compartment, reducing the temperature of the liquid without the need for manual addition of ice cubes. The liquid in the liquid storage compartment can be connected to the liquid storage bag through the connection pipe, allowing the liquid to enter the liquid storage bag and then use the liquid storage bag for ice therapy, which is very convenient to use.

### BRIEF DESCRIPTION OF DRAWINGS

FIG. 1 is a perspective view of a therapeutic device for cold or hot compress provided in this application.

FIG. 2 is a perspective view of a partial structure of the therapeutic device for cold or hot compress provided in this application.

FIG. 3 is a plan view of a liquid storage bag of the therapy device for cold or hot compress provided in this application.

Numeral reference: 1- box body, 2- connection pipe, 3- refrigeration component, 4- quick connector, 5- liquid pump, 6- liquid storage bag, 1a- liquid storage compartment, 2a- first pipeline, 2b- second pipeline.

### DESCRIPTION OF EMBODIMENTS

In order to make the purpose, technical solution, and advantages of this application clearer and more understandable, the following will provide further detailed explanations of this application in combination with the accompanying drawings and embodiments. It should be understood that specific embodiments described herein are only used to explain the present application and are not intended to limit the present application.

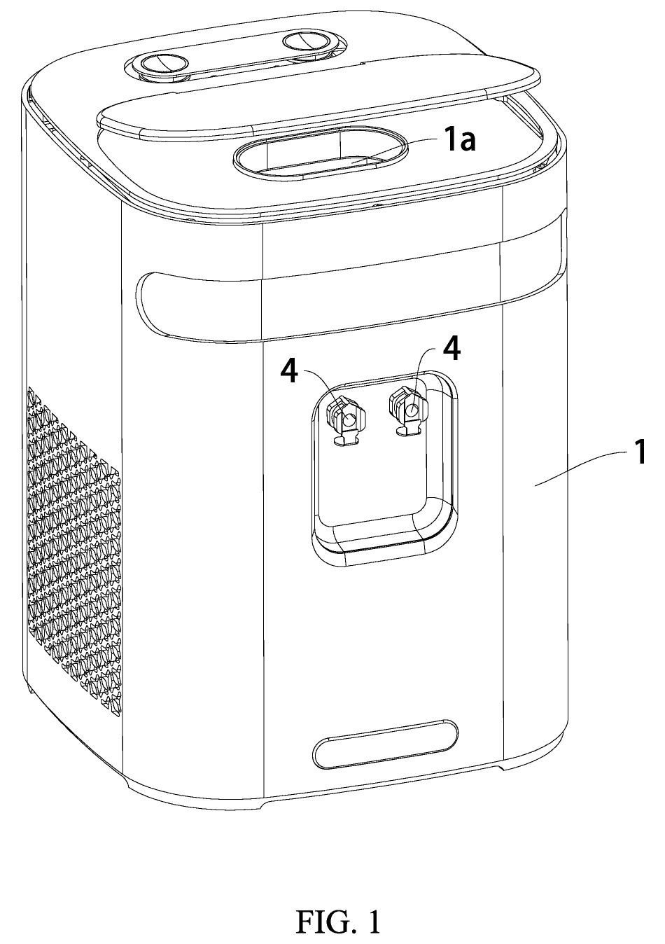

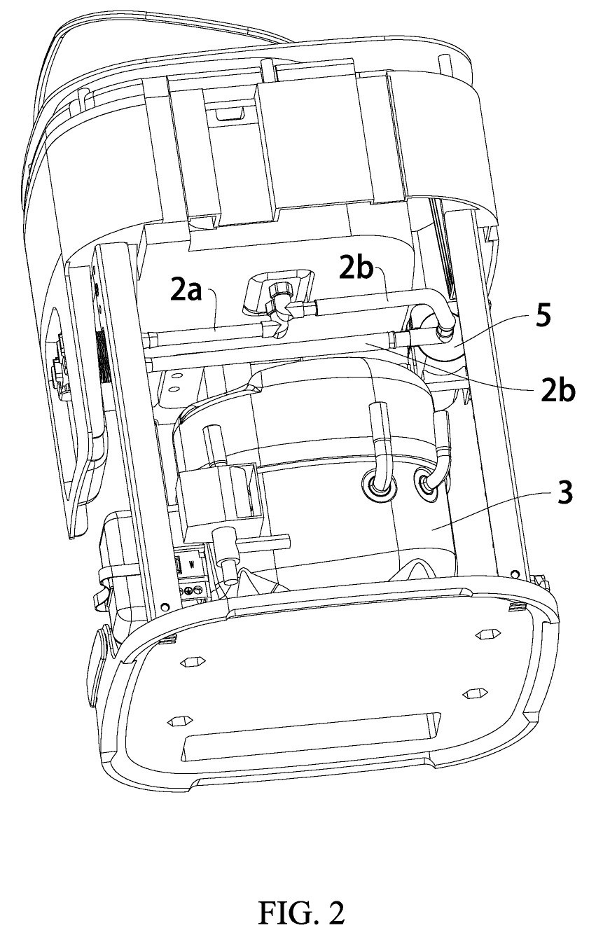

Please refer to FIG. 1 and 2. This embodiment provides a therapeutic device for cold or hot compress, including a box body 1, a connection pipe 2, and a refrigeration component 3. The box body 1 serves as a main body to carry other components, the connection pipe 2 is configured to transport liquid, and the refrigeration component 3 is configured to cool the liquid.

A liquid storage compartment 1a is formed inside the box body 1, and the liquid storage compartment 1a stores the liquid. The liquid in the liquid storage compartment 1a can generally be water or a liquid with a higher specific heating capacity, but for cost and safety considerations, water is generally used. The refrigeration component 3 is configured to reduce temperature of the liquid in the liquid storage compartment 1a. An opening is provided on a top side of the box body 1 for manually adding liquid to the liquid storage compartment 1a.

The refrigeration principle of the refrigeration component 3 is similar to that of a refrigerator air conditioner. The refrigeration working principle of the refrigerator is mainly based on a circulation process of the refrigerant, and the refrigeration effect is achieved through components such as a compressor, a condenser, an evaporator, and a throttling device. The core component of the refrigerator is the compressor, which provides mechanical energy through an electric motor to drive the refrigerant to circulate in the system. The refrigerant absorbs heat during evaporation, thereby achieving the effect of cooling. A specific process is as follows: 1. A compression process: the compressor sucks in low-temperature and low-pressure refrigerant gas, and it is compressed into high-temperature and high-pressure gas. 2. A condensation process: when the high-temperature and high-pressure refrigerant gas passes through the condenser, it exchanges heat with the external environment, releases heat, and becomes liquid. 3. A throttling process: liquid refrigerant is throttled through capillary tubes or expansion valves, reducing pressure and temperature. 4. An evaporation process: low temperature and low-pressure refrigerant is evaporated in the evaporator, absorbs heat from the refrigerator, and then returns to the compressor to complete a cycle. In this embodiment, the refrigeration component 3 includes an evaporator, a compressor, and a condenser, the evaporator is provided in the liquid storage compartment 1a. In order to ensure that the liquid temperature in the liquid storage compartment 1a is as stable as possible, it is generally necessary to install a temperature sensor in the liquid storage compartment 1a and control the operation of the refrigeration component 3 through a control circuit. The control logic and principle can be based on existing ones. Additionally, it should be noted that air conditioning also has heating functions. By using the heating cycle, the temperature of the liquid in the liquid storage compartment can be raised. However, due to the principal limitation, when the temperature needs to be raised relatively high, energy consumption is high, which is not conducive to practical application. In this case, electric heating can be used for heating. In an implementation mode, the therapy device for cold or hot compress further includes a heating element, and the heating element is configured to heat the liquid in the liquid storage compartment 1a. The heating element can be an electric heating wire. After the liquid in the liquid storage compartment 1a is heated to a certain temperature, hot compress can be performed.

The connection pipe 2 is communicated to the liquid storage compartment 1a for transporting liquid in and out of the liquid storage compartment 1a. In an implementation mode, the connection pipe 2 includes a first pipeline 2a and a second pipeline 2b, one end of the first pipeline 2a is communicated to the liquid storage compartment 1a and one end of the second pipeline 2b is communicated to the liquid storage compartment 1a. The first pipeline 2a and the second pipeline 2b are respectively used for input and output of liquid. In an implementation mode, the first pipeline 2a is configured to input liquid into the liquid storage compartment 1a, and the second pipeline 2b is configured to output liquid from the liquid storage compartment 1a. In an implementation mode, the therapy device for cold or hot compress further includes two first quick connectors 4. The other end of the first pipeline 2a is connected to one of the two first quick connectors 4, and the other end of the second pipeline 2b is connected to the other one of the two first quick connectors 4. The two first quick connectors 4 can use a common Colder Products Company, (CPC) connector. The structure of the quick connectors 4 can prevent the liquid in the pipeline connected to it from flowing out when it is not connected, and the liquid in the pipeline can only flow after the first quick connectors 4 are connected to second quick connectors 4. In an implementation mode, the therapy device for cold or hot compress further includes a liquid pump 5, and the liquid pump 5 is connected to the second pipeline 2b.

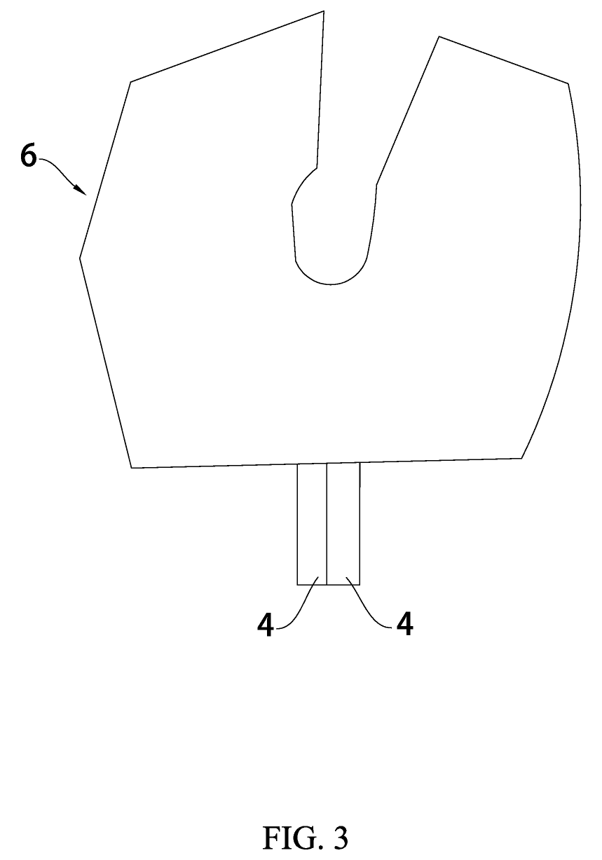

Please refer to FIG. 3. In an implementation mode, the therapeutic device for cold or hot compress further includes a liquid storage bag 6, and the liquid storage bag 6 is connected to two second quick connectors 4. The two second quick connectors 4 connected to the liquid storage bag 6 can be respectively connected to the two first quick connectors 4 of the first pipeline 2a and the second pipeline 2b, so that both the first pipeline 2a and the second pipeline 2b are communicated to the liquid storage bag 6. After the liquid storage bag 6 is communicated to the first pipeline 2a and the second pipeline 2b, the liquid pump 5 can pump the liquid in the liquid storage compartment into the liquid storage bag 6. The original liquid in the liquid storage bag will be squeezed out and enter the first pipeline 2a from the other quick connector 4, thereby entering the liquid storage compartment 1a.

In summary, the present application provides a therapeutic device for cold or hot compress, which uses the refrigeration component 3 to exchange temperature with the liquid in the liquid storage compartment 1a, reducing the temperature of the liquid without the need for manual addition of ice cubes. The liquid in the liquid storage compartment 1a can be communicated to the liquid storage bag through the connection pipe 2, allowing the liquid to enter the liquid storage bag and then use the liquid storage bag for ice therapy, which is very convenient to use.

The specific implementation methods of the present application do not constitute a limitation on the protection scope of the present application. Any other corresponding changes and modifications made based on the technical concept of this application shall be included within the protection scope of the claims of this application.

What is claimed is:

1. A therapeutic device for cold or hot compress, comprising a box body, a connection pipe, and a refrigeration component,
wherein a liquid storage compartment is formed inside the box body, and liquid is configured to be stored in the liquid storage compartment; the refrigeration component is configured to reduce temperature of the liquid in the liquid storage compartment;
the connection pipe is configured to communicate with the liquid storage compartment and transport liquid in and out of the liquid storage compartment.

2. The therapeutic device for cold or hot compress according to claim 1, wherein the connection pipe comprises a first pipeline and a second pipeline, wherein one end of the first pipeline is communicated to the liquid storage compartment, and one end of the second pipeline is communicated to the liquid storage compartment.

3. The therapeutic device for cold or hot compress according to claim 2, wherein the first pipeline is configured to transport the liquid into the liquid storage compartment, and the second pipeline is configured to transport the liquid from the liquid storage compartment.

4. The therapeutic device for cold or hot compress according to claim 2, wherein the therapeutic device for cold or hot compress further comprises two first quick connectors,
wherein the other end of the first pipeline is connected to one of the two the first quick connectors, and the other end of the second pipeline is connected to the other one of the two first quick connectors.

5. The therapeutic device for cold or hot compress according to claim 4, wherein the therapeutic device for cold or hot compress further comprises a liquid pump that is connected to the second pipeline.

6. The therapeutic device for cold or hot compress according to claim 4, wherein the therapeutic device for cold or hot compress further comprises a liquid storage bag, and the liquid storage bag is connected to two second quick connectors;
the two second quick connectors connected to the liquid storage bag are respectively connected to the two first quick connectors of the first pipeline and the second pipeline, so that both the first pipeline and the second pipeline are connected to the liquid storage bag.

7. The therapeutic device for cold or hot compress according to claim 1, wherein the therapeutic device for cold or hot compress further comprises a heating element configured to heat the liquid in the liquid storage compartment.

8. The therapeutic device for cold or hot compress according to claim 1, wherein an opening is provided on a top side of the box body configured to add liquid to the liquid storage compartment.

9. The therapeutic device for cold or hot compress according to claim 1, wherein the refrigeration component comprises an evaporator, a compressor, and a condenser, and the evaporator is provided in the liquid storage compartment.
